(19) United States
(12) Patent Application Publication
(10) Pub. No.: US 2017/0251934 A1
(43) Pub. Date: Sep. 7, 2017

(54) PULSE WAVE MEASUREMENT DEVICE AND BLOOD PRESSURE MEASUREMENT DEVICE

(71) Applicant: NEC Corporation, Tokyo (JP)

(72) Inventors: Yuji OHNO, Tokyo (JP); Masahiro KUBO, Tokyo (JP); Katsumi ABE, Tokyo (JP); Kimiyasu TAKOH, Tokyo (JP); Ersin ALTINTAS, Tokyo (JP)

(73) Assignee: NEC Corporation, Tokyo (JP)

(21) Appl. No.: 15/507,177

(22) PCT Filed: Aug. 19, 2015
(86) PCT No.: PCT/JP2015/004146

§ 371 Date: Feb. 27, 2017

(30) Foreign Application Priority Data
Aug. 27, 2014 (JP) 2014-172301

(51) Int. Cl. A61B 5/021; A61B 5/00

(52) U.S. Cl. CPC A61B 5/02108; A61B 5/725; A61B 5/6824; A61B 5/7275; A61B 2562/0219; A61B 5/024

(57) ABSTRACT

A pulse wave measurement device includes an acceleration sensor that detects a vibration and a vibration transmitter that transmits a vibration caused by pulsation in a measured site, wherein a length of the vibration transmitter in a specific direction is longer than a length of the acceleration sensor in a longitudinal direction.

### TECHNICAL FIELD

The present invention relates to a pulse wave measurement device and a blood pressure measurement device equipped with the same.

### BACKGROUND ART

Blood pressure is one of the most important information items for monitoring human health conditions. Blood pressure includes systolic blood pressure (also known as maximal pressure) and diastolic blood pressure (also known as minimal pressure). In recent years, systolic or diastolic blood pressure has been used as an indicator contributing to risk analyses on cardiovascular diseases including stroke, heart failure, and myocardial infarction.

One known method for measuring blood pressure is the oscillometric method, which uses a cuff to apply pressure on an upper arm to measure blood pressure. According to the oscillometric method, the amplitude of a pulse wave measured on an upper arm changes with varying degrees of pressure in the cuff. Based on the amplitude of a pulse wave, the blood pressure during systole (i.e., systolic blood pressure or maximal pressure) as well as the blood pressure during diastole (i.e., diastolic blood pressure or minimal pressure) are measured. Hence, to estimate blood pressure using pulse wave information, a correct pulse wave has to be obtained.

As a technology for measuring a pulse wave, PTL 1 describes a blood pressure measurement device based on a double-cuff system that includes an occluding air bag for pressing a blood vessel and an air bag for detecting a pulse wave. In the double-cuff system, a pulse wave is detected in a central portion under the pulse wave detecting air bag, which is separated from the function of occluding blood flow. The blood pressure measurement device according to PTL 1 involves a complex device configuration for measurement of true pulse waves, requiring complex controls on the individual air bags.

PTL 2 describes a blood pressure measurement device that includes a vibration sensor to be positioned on an artery to measure a pulse wave. The blood pressure measurement device according to PTL 2 involves a complex device configuration for the positioning, failing to obtain a true pulse wave once the vibration sensor is displaced out of the measured site by movement of the subject.

PTL 3 describes a pulse wave measurement device equipped with a plurality of sensors. The pulse wave measurement device includes a vibration membrane that transmits a displacement of skin surface caused by a pulse wave, a frame that fixes the outer edge of the vibration membrane, and partitions that separate a central portion of the vibration membrane into a plurality of sections. The pulse wave measurement device further includes a plurality of sensor elements that are arranged on the vibration membrane within the plurality of sections and that transform a vibration of the vibration membrane into an electric signal.

In the pulse wave measurement device according to PTL 3, the vibration membrane is separated into sections of sensor elements with partitions, and thus stresses and displacements transmitted to the individual sensor elements are separated from and independent of one another. This reduces crosstalk between adjacent sensor elements arising from pressure or stress on the vibration membrane, achieving measurement with higher precision. In addition, since the plurality of sensor elements are two-dimensionally arranged, any pinpoint site for detecting a pulse wave can be covered by any of the plurality of sensor elements to pick up the pulse wave.

### CITATIONS LIST

### Patent Literature

[PTL 1] Japanese Patent No. 4819594

[PTL 2] Japanese Patent No. 3873625

[PTL 3] Japanese Unexamined Patent Application Publication No. 2011-072645

[PTL 4] Japanese Unexamined Patent Application Publication No. 2005-156531

### SUMMARY OF INVENTION

### Technical Problem

The pulse wave measurement device disclosed in PTL 3 includes a lot of sensor elements that are needed to be two-dimensionally arranged to extend the range of pulse wave detection. In addition, a vibration of the vibration membrane in the pulse wave measurement device is limited to the individual sections of the sensor elements, which prevents the vibration membrane from vibrating to a greater extent, resulting in a lower detection sensitivity to vibrations.

Accordingly, an object of the present invention is to provide a pulse wave measurement device that is capable of extending the range of pulse wave detection with a simple configuration and achieving accurate pulse wave measurement, as well as to provide a blood pressure measurement device equipped with such a pulse wave measurement device.

### Solution to Problem

A pulse wave measurement device according to one aspect of the present invention includes an acceleration sensor that detects a vibration and a vibration transmitting unit that transmits a vibration caused by pulsation in a measured site, wherein a length of the vibration transmitting unit in a specific direction is longer than a length of the acceleration sensor in a longitudinal direction.

A blood pressure measurement device according to one aspect of the present invention includes the pulse wave measurement device.

### Advantageous Effects of Invention

The present invention can provide a pulse wave measurement device that is capable of extending the range of pulse wave detection with a simple configuration and achieving accurate pulse wave measurement, as well as a blood pressure measurement device equipped with such a pulse wave measurement device.

### BRIEF DESCRIPTION OF DRAWINGS

FIG. 1 illustrates a configuration of a pulse wave measurement device according to a first example embodiment.

FIG. 2 illustrates examples of an acceleration sensor according to the first example embodiment.

FIG. 3 illustrates examples of a vibration transmitting unit according to the first example embodiment.

FIG. 4 illustrates examples of a positionally relationship between the acceleration sensor and the vibration transmitting unit according to the first example embodiment.

FIG. 5 illustrates an example of a positionally relationship between the acceleration sensor and the vibration transmitting unit according to the first example embodiment.

FIG. 6 presents a comparison example illustrating a positionally relationship between the acceleration sensor and the measured site.

FIG. 7 illustrates a positionally relationship between the pulse wave measurement device and the measured site according to the first example embodiment.

FIG. 8 illustrates a state in which the acceleration sensor is attached on an upper arm.

FIG. 9 illustrates pulse wave signals obtained with the acceleration sensor.

FIG. 10 is a diagram representing pulse wave measurement device of the first example embodiment, the device being attached on an upper arm.

FIG. 11 illustrates pulse wave signals obtained with the pulse wave measurement device according to the first example embodiment.

FIG. 12 illustrates a configuration of a pulse wave measurement device according to a second example embodiment.

FIG. 13 illustrates a configuration of a pulse wave measurement device according to a third example embodiment.

FIG. 14 illustrates a configuration of a pulse wave measurement device according to a fourth example embodiment.

FIG. 15 illustrates a configuration of a pulse wave measurement device according to a fifth example embodiment.

FIG. 16 illustrates a configuration of a pulse wave measurement device according to a sixth example embodiment.

FIG. 17 is a block diagram illustrating a configuration of a blood pressure measurement device according to a seventh example embodiment.

FIG. 18 is a block diagram illustrating a configuration of a blood pressure measurement device according to a variation of the seventh example embodiment.

FIG. 19 is a block diagram illustrating a configuration of a blood pressure measurement device according to a variation of the seventh example embodiment.

FIG. 20 is a schematic diagram illustrating a configuration of a watch according to an eighth example embodiment.

FIG. 21 is a block diagram illustrating a hardware configuration for implementing with a computer either the pressure control unit or the blood pressure estimating unit included in the blood pressure measurement device according to the seventh example embodiment.

### DESCRIPTION OF EMBODIMENTS

Example embodiments of the present invention will now be described with reference to the drawings.

### First Example Embodiment

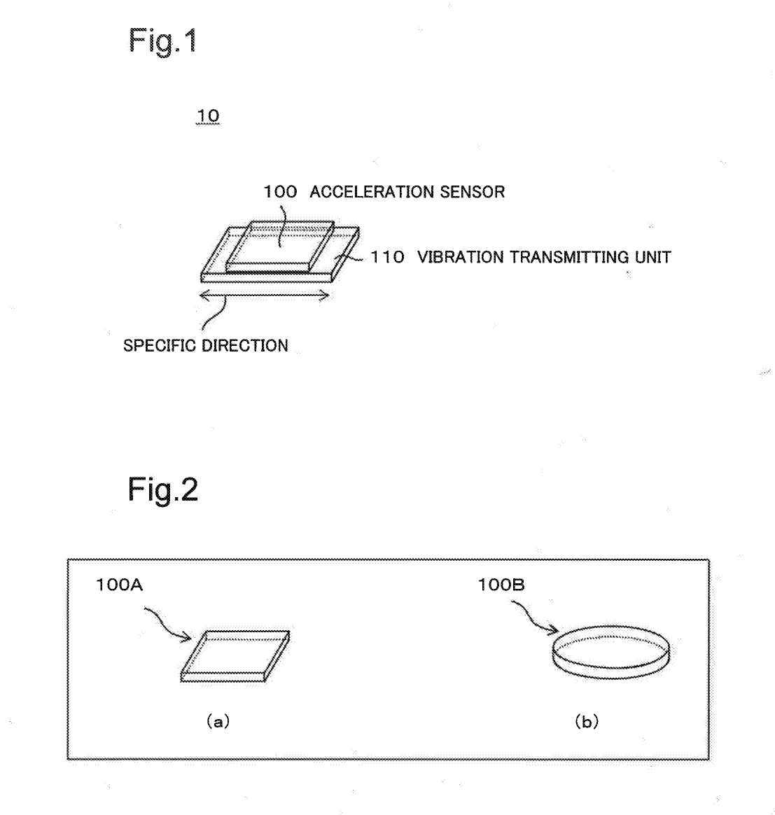

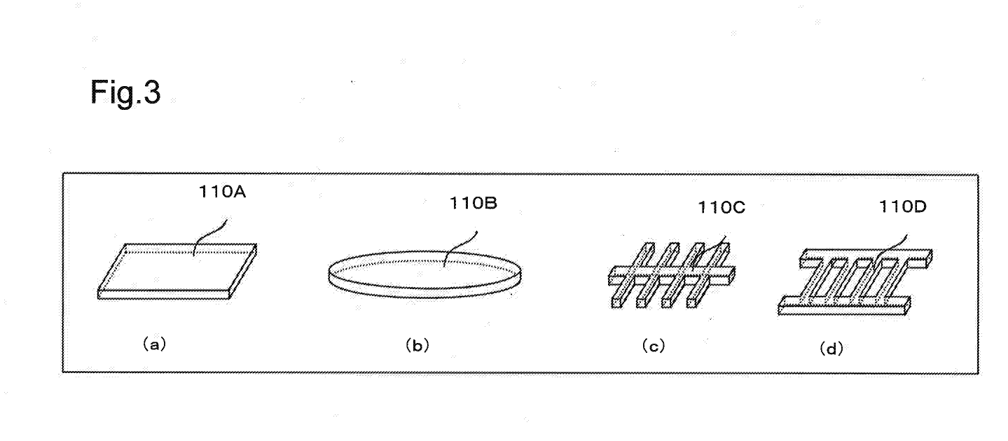

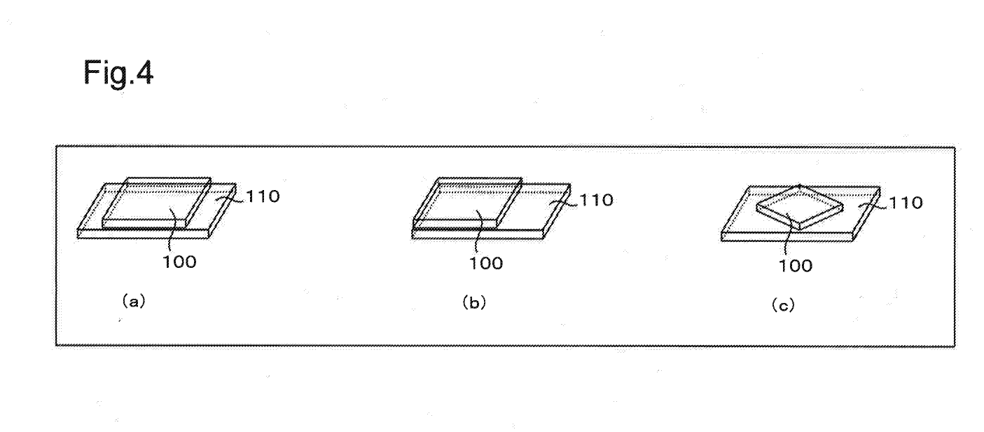

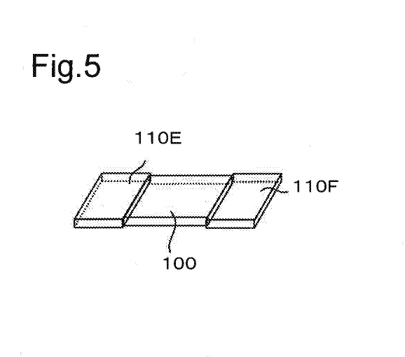

A first example embodiment is described below. FIG. 1 illustrates a configuration of a pulse wave measurement device according to the first example embodiment. FIG. 2 illustrates examples of an acceleration sensor according to the first example embodiment. FIG. 3 illustrates examples of a vibration transmitting unit according to the first example embodiment. FIG. 4 illustrates examples of a positionally relationship between the acceleration sensor and the vibration transmitting unit according to the first example embodiment. FIG. 5 illustrates an example of a positionally relationship between the acceleration sensor and the vibration transmitting unit according to the first example embodiment.

As illustrated in FIG. 1, the pulse wave measurement device 10 according to the first example embodiment includes the acceleration sensor 100 and the vibration transmitting unit 110. A configuration where the length of the vibration transmitting unit 110 in a specific direction is longer than the length of the acceleration sensor 100 in the longitudinal direction is employed. It is assumed that the specific direction is along the longest dimension of the vibration transmitting unit 110.

The acceleration sensor 100 detects a vibration in a measured site and transforms the vibration information into an electrical signal. The resulting electrical signal is transmitted to the outside through wiring (not illustrated). When transforming into an electrical signal, a band-pass filter that passes a specific range of frequencies, an adaptive filter, or a Kalman filter may be applied. Such filtering can produce electrical signals free from noises except vibration information regarding pulse waves. Instead of using wiring, electrical signals may also be transmitted by a wireless unit (not illustrated) to the outside of the acceleration sensor 100 in the form of wireless signals. The band-pass filter and the wireless unit may be configured to be built in the acceleration sensor 100 or to be added to the acceleration sensor 100.

The acceleration sensor 100 is not limited to a uniaxial acceleration sensor, a biaxial acceleration sensor, or a triaxial acceleration sensor. As a sensing method for detecting acceleration, electrostatic type, piezoelectric type, resistance type, thermo-fluid type, electrodynamic type, servo type, or magnetic type sensing may be applied. A sensing method other than those listed above may also be applied.

The shape of the acceleration sensor 100 may be rectangular, as represented by the acceleration sensor 100A in (a) of FIG. 2, or may be circular, as represented by the acceleration sensor 100B in (b) of FIG. 2. The acceleration sensor may be in a shape other than the aforementioned ones.

The vibration transmitting unit 110 has a function to transmit a vibration that has been caught at some point to the entire vibration transmitting unit 110. Materials that may be used for the vibration transmitting unit 110 include, for example, a metal (e.g., aluminum, copper, or aluminum alloy), a resin (e.g., polyethylene, polypropylene, polystyrene, or polyvinyl chloride), or a liquid (including a gel). The vibration transmitting unit 110 may be a sealed bag containing a gas, a liquid, or a solid.

The vibration transmitting unit 110 may be in any shape as long as the length of the vibration transmitting unit 110 in the specific direction is longer than the length of the acceleration sensor 100 in the longitudinal direction. For example, the vibration transmitting unit may be any of a rectangular vibration transmitting unit 110A illustrated in (a) of FIG. 3, an elliptical vibration transmitting unit 110B illustrated in (b) of FIG. 3, a comb-shaped vibration transmitting unit 110C illustrated in (c) of FIG. 3, and a ladder-shaped vibration transmitting unit 110D illustrated in (d) of FIG. 3. The shape of the vibration transmitting unit 110 is not limited to these ones. Although no specific limit is imposed on the thickness of the vibration transmitting unit 110, the thickness is preferably 5 mm or less.

The acceleration sensor 100 and the vibration transmitting unit 110 are adhered to each other by, for example, applying double-sided tape on both the acceleration sensor 100 and the vibration transmitting unit 110 to stick them together. The sticking may be based on an adhesive, heat welding, or ultrasonic welding.

Regarding a positionally relationship between the acceleration sensor 100 and the vibration transmitting unit 110, it is preferable that the acceleration sensor 100 is placed near the center of the length of the vibration transmitting unit 110 in the specific direction, as illustrated in (a) of FIG. 4. In this case, the acceleration sensor 100 can detect a vibration through the vibration transmitting unit 110 at a higher detection sensitivity. Alternatively, the acceleration sensor 100 may be placed near an end of the vibration transmitting unit 110, as illustrated in (b) of FIG. 4. If each of the acceleration sensor 100 and the vibration transmitting unit 110 has its ends as illustrated in (c) of FIG. 4, an end of the acceleration sensor 100 may not necessarily be parallel to an end of the vibration transmitting unit 110.

Alternatively, vibration transmitting units 110E and 110F may be placed on end faces of the acceleration sensor 100, as illustrated in FIG. 5. In FIG. 5, the vibration transmitting units 110 are placed on two opposing ends of the acceleration sensor 100. The vibration transmitting unit 110 may be placed not only as illustrated in FIG. 5 but on one or a plurality of end faces.

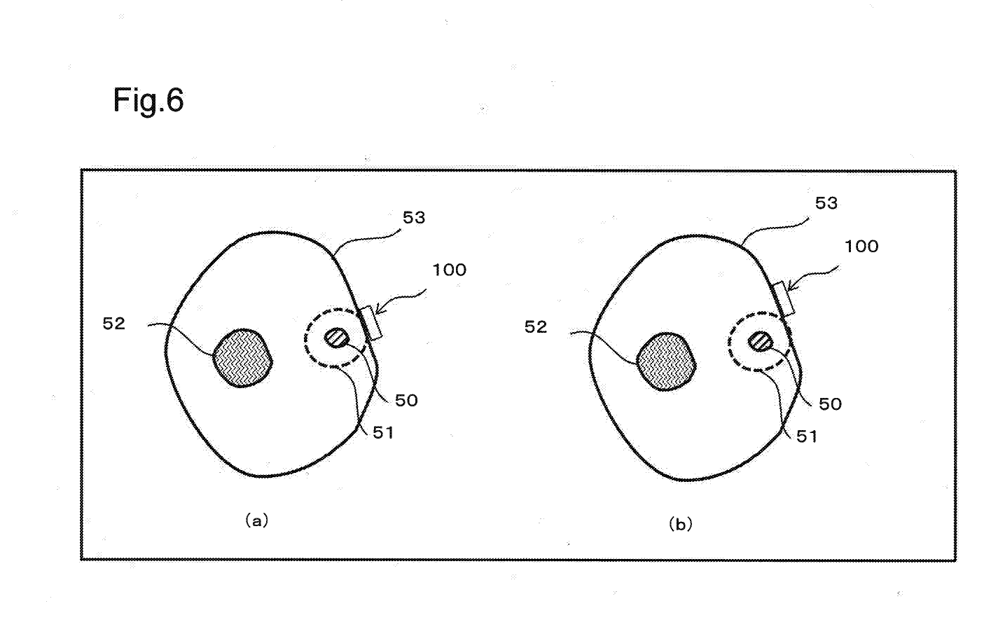

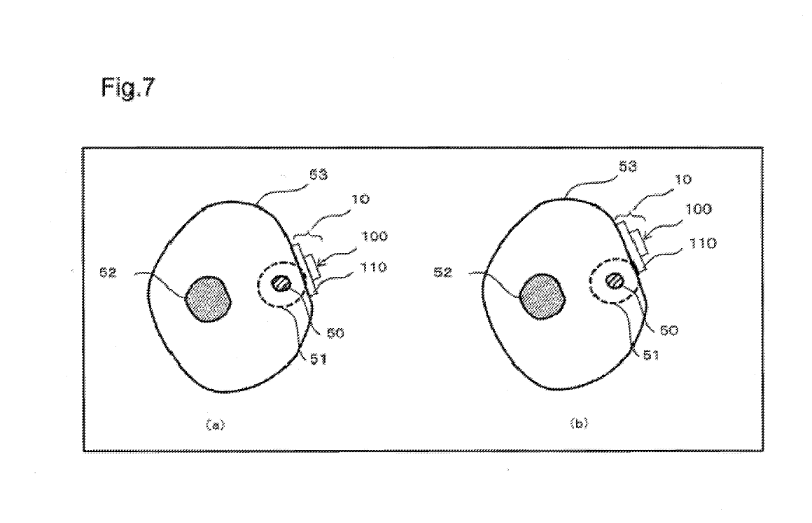

The following describes operations of the pulse wave measurement device 10. FIG. 6 presents a comparison example illustrating a positionally relationship between the acceleration sensor and the measured site. FIG. 7 illustrates a positionally relationship between the pulse wave measurement device according to the first example embodiment and the measured site. FIGS. 6 and 7 present cross-sectional views each illustrating the acceleration sensor 100 placed near a measured site on an artery 50. FIGS. 6 and 7 represent a case where a pulse wave is measured on an upper arm, and schematically illustrate the upper arm, i.e., the measured site, with a bone 52, an artery 50, and a surface layer 53.

As illustrated in (a) of FIG. 6, the acceleration sensor 100 is placed on the surface layer 53 near the artery 50. In this case, the acceleration sensor 100 is located within a vibration transmission range 51, and thus a vibration on the surface layer of skin caused by pulsation is obtained. On the other hand, when the acceleration sensor 100 is placed on the surface layer 53 out of the vibration transmission range 51 as illustrated in (b) of FIG. 6, the acceleration sensor 100 cannot detect a vibration of the artery 50.

As illustrated in (a) of FIG. 7, the pulse wave measurement device 10 of the first example embodiment is placed on the surface layer 53 near the artery 50. In this case, the acceleration sensor 100 is located within the vibration transmission range 51, and thus a vibration on the surface layer of skin caused by pulsation is easily obtained.

On the other hand, suppose that the acceleration sensor 100 in the pulse wave measurement device 10 of the first example embodiment is placed on the surface layer 53 out of the vibration transmission range 51, as illustrated in (b) of FIG. 7. In this case, although the acceleration sensor 100 in the pulse wave measurement device 10 is located out of the vibration transmission range 51, the vibration transmitting unit 110, which is located within the vibration transmission range 51, transmits a vibration of the surface layer caused by pulsation to the acceleration sensor 100. Accordingly, the acceleration sensor 100 in (b) of FIG. 7 can detect a vibration caused by pulsation.

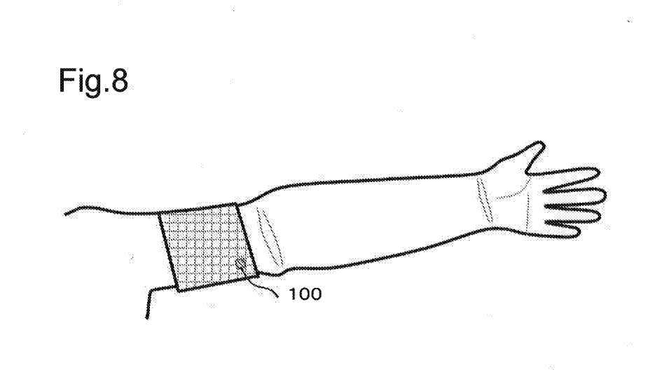

FIG. 8 conceptually illustrates a state in which the acceleration sensor 100 is directly attached on an upper arm.

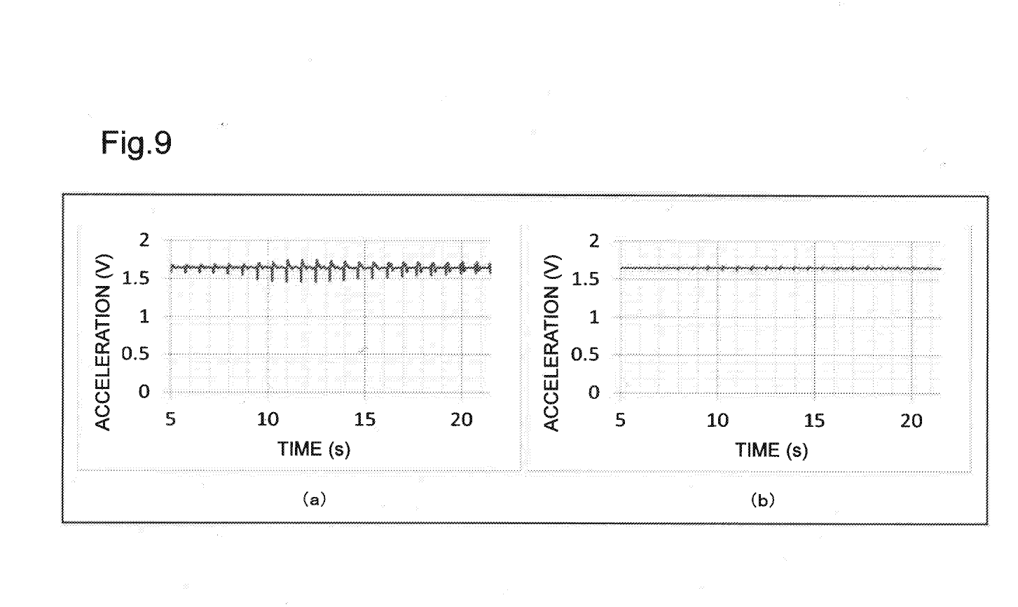

FIG. 9 represents time-series pulse waves detected by the acceleration sensor 100, when a varying compressing (pressurizing) pressure is applied to the upper arm as in the state illustrated in FIG. 8. The graph in (a) of FIG. 9 indicates a pulse wave signal obtained with the acceleration sensor 100 placed on the surface layer 53 near the artery 50, while the graph in (b) of FIG. 9 indicates a pulse wave signal obtained with the acceleration sensor 100 misaligned with the artery 50. As indicated in (a) of FIG. 9, a clear pulse wave signal can be detected with the acceleration sensor 100 placed near the artery 50. On the other hand, in the case of misalignment, a pulse wave signal can be detected only slightly as indicated in (b) of FIG. 9.

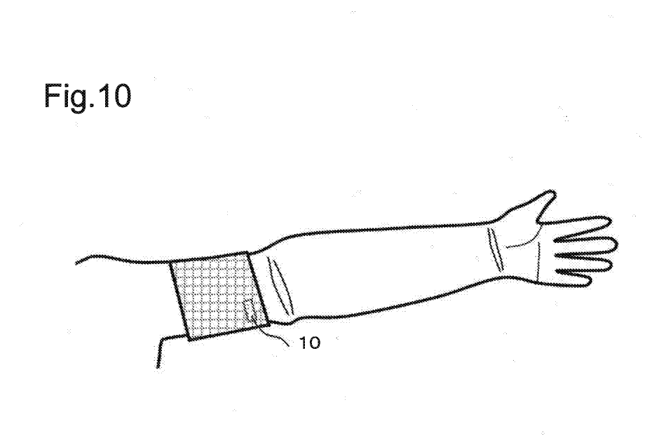

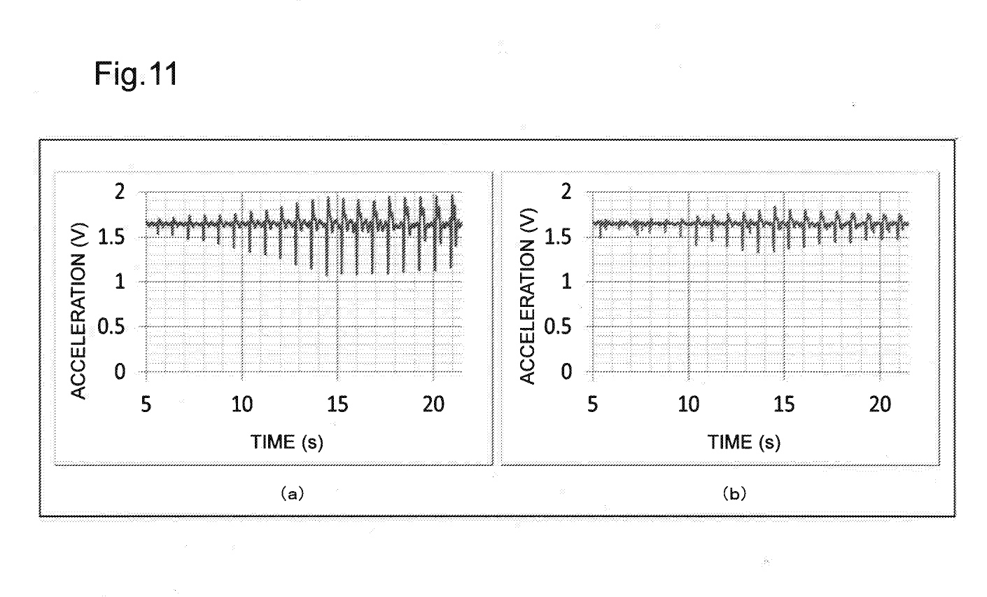

FIG. 10 conceptually illustrates a state in which the pulse wave measurement device 10 of the first example embodiment is attached on an upper arm. FIG. 11 represents a relationship between a varying compressing (pressurizing) pressure applied to the upper arm and a time-series pulse wave signal detected by the pulse wave measurement device 10 as in the state illustrated in FIG. 10. The graph in (a) of FIG. 11 indicates a pulse wave obtained with the acceleration sensor placed on the surface layer 53 near the artery 50, while the graph in (b) of FIG. 11 indicates a pulse wave obtained with the acceleration sensor misaligned with the artery 50. As indicated in FIG. 11, the pulse wave measurement device 10 of the first example embodiment can detect a pulse wave signal even when the acceleration sensor is misaligned with the artery 50.

As seen above, in the pulse wave measurement device of the first example embodiment, the vibration transmitting unit can catch a vibration caused by pulsation and the acceleration sensor can detect a vibration transmitted by the vibration transmitting unit, irrespective of whether the acceleration sensor is placed on a surface layer out of the transmission range of vibration of an artery during the pulse wave measurement. Hence, the range of pulse wave detection can be extended with a simple configuration, and a pulse wave can be measured accurately. This is because the pulse wave measurement device includes the acceleration sensor and the vibration transmitting unit, and employs a configuration where the length of the vibration transmitting unit in the specific direction is longer than the length of the acceleration sensor in the longitudinal direction.

### Second Example Embodiment

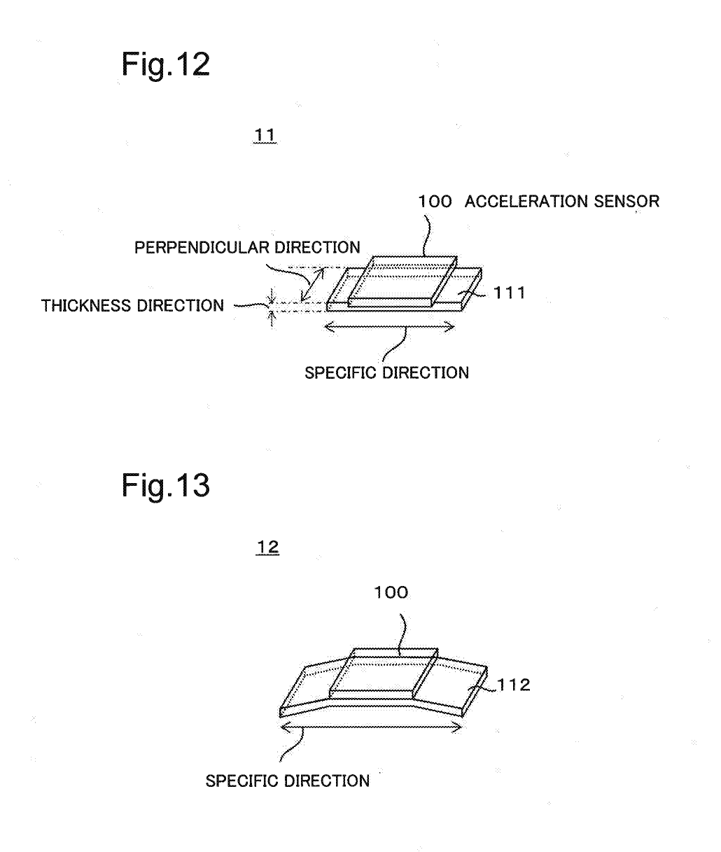

A second example embodiment will now be described. FIG. 12 illustrates a configuration of a pulse wave measurement device according to the second example embodiment. In FIG. 12, identical symbols are given to components similar to those in the first example embodiment.

As illustrated in FIG. 12, the pulse wave measurement device 11 according to the second example embodiment includes the acceleration sensor 100 and the vibration transmitting unit 111. As with the first example embodiment, the length of the vibration transmitting unit 111 in the specific direction is longer than the length of the acceleration sensor 100 in the longitudinal direction. In addition, in the pulse wave measurement device 11 of the second example embodiment, the length of the vibration transmitting unit 111 in the direction perpendicular (hereinafter referred to as the perpendicular direction) to the specific direction and to the thickness direction is equal to or less than the length of the acceleration sensor 100. This indicates that the acceleration sensor 100 may not entirely be coupled to the vibration transmitting unit 111 of the second example embodiment when a vibration caused by pulsation is transmitted from the vibration transmitting unit 111 to the acceleration sensor 100.

As with the first example embodiment, the vibration transmitting unit 111 has a function to transmit a vibration that has been caught at some point of the vibration transmitting unit 111 to the entire vibration transmitting unit 111. Materials similar to those listed in the first example embodiment may be applied to the vibration transmitting unit 111. The vibration transmitting unit 111 may be in any shape as long as the length of the vibration transmitting unit 111 in the specific direction is longer than the length of the acceleration sensor 100 in the longitudinal direction, and the length of the vibration transmitting unit 111 in the perpendicular direction is equal to or less than the length of the acceleration sensor 100. As with the first example embodiment, the shape of the vibration transmitting unit 111 may be, for example, any of the ones illustrated in (a) to (d) of FIG. 3. The vibration transmitting unit 111 may be in a shape other than the ones illustrated in (a) to (d) of FIG. 3. Although no specific limit is imposed on the thickness of the vibration transmitting unit 111, the thickness is preferably 5 mm or less.

As with the first example embodiment, no specific limitation is imposed on how to couple the acceleration sensor 100 and the vibration transmitting unit 111 to each other. However, if the area of contact between the acceleration sensor 100 and the vibration transmitting unit 111 of the second example embodiment is smaller than that of the first example embodiment, they are preferably stuck together with greater adhesion compared with the first example embodiment.

Regarding a positionally relationship between the acceleration sensor 100 and the vibration transmitting unit 111, as with the first example embodiment, the acceleration sensor 100 is preferably placed near the center of the length of the vibration transmitting unit 111 in the specific direction so as to have a higher detection sensitivity to vibrations. As with the first example embodiment, the acceleration sensor 100 may be placed near an end of the vibration transmitting unit 111, and an end of the acceleration sensor 100 may not necessarily be parallel to an end of the vibration transmitting unit 111. In addition, as illustrated in FIG. 5, the vibration transmitting unit 111 may be placed on one or a plurality of ends of the acceleration sensor 100.

As seen above, the pulse wave measurement device 11 of the second example embodiment can extend the range of pulse wave detection with a simple configuration and can accurately measure a pulse wave. This is because, in the pulse wave measurement device 11 of the second example embodiment, the length of the vibration transmitting unit in the specific direction is longer than the length of the acceleration sensor in the longitudinal direction, as with the pulse wave measurement device 10 of the first example embodiment. In other words, the vibration transmitting unit 111 can transmit a vibration caused by pulsation to the acceleration sensor 100, the vibration being caused in a region of the vibration transmitting unit where the acceleration sensor 100 is not placed.

In addition, in the pulse wave measurement device 11 of the second example embodiment, the length in the perpendicular direction is equal to or less than the length of the acceleration sensor 100, which makes it possible to catch a vibration caused by pulsation in a more limited range in the blood flow direction. Therefore, the second example embodiment achieves detection of a more accurate pulse wave than the first example embodiment.

### Third Example Embodiment

A third example embodiment will now be described. FIG. 13 illustrates a configuration of a pulse wave measurement device according to the third example embodiment. Identical symbols are given to components similar to those in the first example embodiment.

As illustrated in FIG. 13, the pulse wave measurement device 12 according to the third example embodiment includes the acceleration sensor 100 and the vibration transmitting unit 112. The length of the vibration transmitting unit 112 in the specific direction is longer than the length of the acceleration sensor 100 in the longitudinal direction. In addition, the vibration transmitting unit 112 has a curved structure along the specific direction.

As with the first example embodiment, the vibration transmitting unit 112 has a function to transmit a vibration that has been caught at some point of the vibration transmitting unit 112 to the entire vibration transmitting unit 112. Materials similar to those listed in the first example embodiment may be applied to the vibration transmitting unit 112. The vibration transmitting unit 112 may be in any shape as long as the length of the vibration transmitting unit 112 in the specific direction is longer than the length of the acceleration sensor 100 in the longitudinal direction, and the vibration transmitting unit 112 is in curved structure along the specific direction of the vibration transmitting unit 112. As with the first example embodiment, the shape may be, for example, any of the ones illustrated in (a) to (d) of FIG. 3. The shape is not limited to these ones. Although no specific limit is imposed on the thickness, the thickness is preferably 5 mm or less.

The curved shape of the vibration transmitting unit 112 along the specific direction may be a smooth arc or an angulated arc. The radius of curvature of the vibration transmitting unit 112 along the specific direction preferably falls within the range of 1.6 to 8.0 cm.

As with the first example embodiment, no specific limitation is imposed on how to couple the acceleration sensor 100 and the vibration transmitting unit 112 to each other. Regarding a positionally relationship between the acceleration sensor 100 and the vibration transmitting unit 112, as with the first example embodiment, the acceleration sensor 100 is preferably placed near the center of the length of the vibration transmitting unit 112 in the specific direction so as to have a higher detection sensitivity to vibrations. As with the first example embodiment, the acceleration sensor 100 may be placed near an end of the vibration transmitting unit 112, and an end of the acceleration sensor 100 may not necessarily be parallel to an end of the vibration transmitting unit 112. In addition, as illustrated in FIG. 5, the vibration transmitting unit 112 may be placed on one or a plurality of ends of the acceleration sensor 100.

As seen above, the pulse wave measurement device 12 of the third example embodiment can extend the range of pulse wave detection with a simple configuration and can accurately measure a pulse wave. This is because, in the pulse wave measurement device 12 of the third example embodiment, the length of the vibration transmitting unit in the specific direction is longer than the length of the acceleration sensor in the longitudinal direction, as with the pulse wave measurement device 10 of the first example embodiment. In other words, the vibration transmitting unit 112 can transmit a vibration caused by pulsation to the acceleration sensor 100, the vibration being caused in a region of the vibration transmitting unit 112 where the acceleration sensor 100 is not placed.

In addition, since the pulse wave measurement device 12 of the third example embodiment is in curved structure along the specific direction, the vibration transmitting unit 120 in the pulse wave measurement device is more adaptive to a measured site to provide a larger area of contact, thereby achieving detection of a more accurate pulse wave compared with the first example embodiment.

### Fourth Example Embodiment

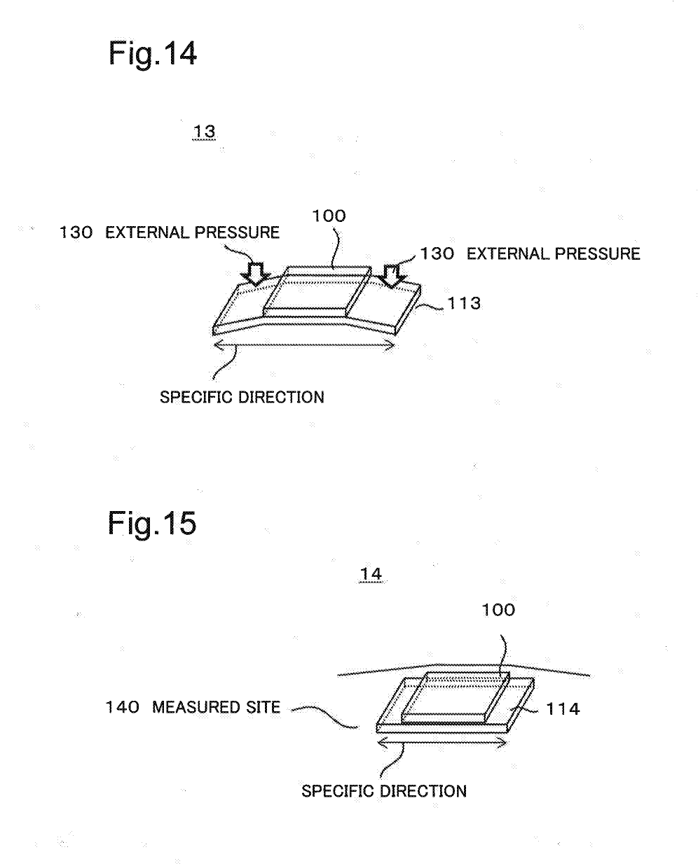

A fourth example embodiment will now be described. FIG. 14 illustrates a configuration of a pulse wave measurement device according to the fourth example embodiment. Identical symbols are given to components similar to those in the first example embodiment.

As illustrated in FIG. 14, the pulse wave measurement device 13 according to the fourth example embodiment includes the acceleration sensor 100 and the vibration transmitting unit 113. The length of the vibration transmitting unit 113 in the specific direction is longer than the length of the acceleration sensor 100 in the longitudinal direction. In addition, the vibration transmitting unit 113 is deformed into a curved shape along the longitudinal direction under an external pressure 130 of 50 mmHg (6666 Pa) or less applied in a direction toward the measured site.

As with the first example embodiment, the vibration transmitting unit 113 has a function to transmit a vibration that has been caught at some point of the vibration transmitting unit 113 to the entire vibration transmitting unit 113.

The vibration transmitting unit 113 is made of a material which is easily deformed under the external pressure 130 of 50 mmHg (6666 Pa) or less, having a Young's modulus of approximately 10 GPa or less. For example, the material may be a sealed bag containing a resin (e.g., polyethylene, polypropylene, polystyrene, or polyvinyl chloride), a liquid (including a gel), or a gas.

The vibration transmitting unit 113 may be in any shape as long as the length of the vibration transmitting unit 113 in the specific direction is longer than the length of the acceleration sensor 100 in the longitudinal direction. As with the first example embodiment, the shape of the vibration transmitting unit 113 may be, for example, any of the ones illustrated in (a) to (d) of FIG. 3. The shape is not limited to these ones. Although no specific limit is imposed on the thickness, the thickness is preferably 5 mm or less.

As with the first example embodiment, no specific limitation is imposed on how to couple the acceleration sensor 100 and the vibration transmitting unit 113 to each other.

Regarding a positionally relationship between the acceleration sensor 100 and the vibration transmitting unit 113, as with the first example embodiment, the acceleration sensor 100 is preferably placed near the center of the length of the vibration transmitting unit 113 in the specific direction so as to have a higher detection sensitivity to vibrations. As with the first example embodiment, the acceleration sensor 100 may be placed near an end of the vibration transmitting unit 113, and an end of the acceleration sensor 100 may not necessarily be parallel to an end of the vibration transmitting unit 113. In addition, as illustrated in FIG. 5, the vibration transmitting unit 113 may be placed on one or a plurality of ends of the acceleration sensor 100.

As seen above, the pulse wave measurement device 13 of the fourth example embodiment can extend the range of pulse wave detection with a simple configuration and can accurately measure a pulse wave. This is because, in the pulse wave measurement device 13 of the fourth example embodiment, the length of the vibration transmitting unit 113 in the specific direction is longer than the length of the acceleration sensor 100 in the longitudinal direction, as with the pulse wave measurement device 10 of the first example embodiment. In other words, the vibration transmitting unit 113 can transmit a vibration caused by pulsation to the acceleration sensor 100, the vibration being caused in a region of the vibration transmitting unit 113 where the acceleration sensor 100 is not placed.

In addition, the vibration transmitting unit 113 in the pulse wave measurement device 13 of the fourth example embodiment is easily deformed under the external pressure 130 of 50 mmHg (6666 Pa) or less. Applying the external pressure 130 to the vibration transmitting unit 113 makes the vibration transmitting unit 113 in the pulse wave measurement device 13 more adaptive to a measured site to provide a larger area of contact, thereby achieving detection of a true pulse wave.

### Fifth Example Embodiment

A fifth example embodiment will now be described. FIG. 15 illustrates a configuration of a pulse wave measurement device according to the fifth example embodiment. Identical symbols are given to components similar to those in the first example embodiment.

As illustrated in FIG. 15, the pulse wave measurement device 14 according to the fifth example embodiment includes the acceleration sensor 100 and the vibration transmitting unit 114. The length of the vibration transmitting unit 114 in the specific direction is longer than the length of the acceleration sensor 100 in the longitudinal direction, and the vibration transmitting unit 114 has a higher vibration transmissibility relative to the measured site 140.

As with the first example embodiment, the vibration transmitting unit 114 has a function to transmit a vibration that has been caught at some point of the vibration transmitting unit 114 to the entire vibration transmitting unit 114. In addition, the vibration transmitting unit 114 has a higher vibration transmissibility relative to the measured site 140 (e.g., skin). Specifically, the shape or material of the vibration transmitting unit 114 represents a vibration transmissibility of 1 or higher at a vibration frequency falling within the range of 0.5 to 2.5 Hz in the vibration transmitting unit 114.

Vibration transmissibility λ, expressed by Equation 1, is the ratio of reaction force at a support point to force inputted from a vibration source:

$$λ = \sqrt{\frac{1 + 4 {ζ}^{2} {β}^{2}}{{( 1 - {β}^{2} )}^{2} + 4 {ζ}^{2} {β}^{2}}} \\ β = \frac{ω}{{ω}_{n}} , ζ = \frac{c}{{c}_{c}} , {c}_{c} = 2 \sqrt{mk} , {ω}_{n} = \sqrt{\frac{k}{m}} \qquad ( EQUATION 1 )$$

where ζ: damping ratio, c: damping coefficient, c_{c}: critical damping coefficient, m: mass, k: spring constant, ω: respective vibration frequency, and ωₙ: natural angular vibration frequency.

The material may be, for example, a sealed bag containing a metal (e.g., aluminum, copper, or aluminum alloy), a resin (e.g., polyethylene, polypropylene, polystyrene, or polyvinyl chloride), or a solid.

The vibration transmitting unit 114 may be in any shape as long as the length of the vibration transmitting unit 114 in the specific direction is longer than the length of the acceleration sensor 100 in the longitudinal direction. As with the first example embodiment, the shape may be, for example, any of the ones illustrated in (a) to (d) of FIG. 3. The shape of the vibration transmitting unit 114 is not limited to these ones. Although no specific limit is imposed on the thickness of the vibration transmitting unit 114, the thickness is preferably 5 mm or less.

As with the first example embodiment, no specific limitation is imposed on how to couple the acceleration sensor 100 and the vibration transmitting unit 114 to each other. Regarding a positionally relationship between the acceleration sensor 100 and the vibration transmitting unit 114, as with the first example embodiment, the acceleration sensor 100 is preferably placed near the center of the length of the vibration transmitting unit 114 in the specific direction so as to have a higher detection sensitivity to vibrations. As with the first example embodiment, the acceleration sensor 100 may be placed near an end of the vibration transmitting unit 114, and an end of the acceleration sensor 100 may not necessarily be parallel to an end of the vibration transmitting unit 114. In addition, as illustrated in FIG. 5, the vibration transmitting unit 114 may be placed on one or a plurality of ends of the acceleration sensor 100.

As seen above, the pulse wave measurement device 11 of the fifth example embodiment can extend the range of pulse wave detection with a simple configuration and can accurately measure a pulse wave. This is because, in the pulse wave measurement device 14 of the fifth example embodiment, the length of the vibration transmitting unit 114 in the specific direction is longer than the length of the acceleration sensor 100 in the longitudinal direction, as with the pulse wave measurement device 10 of the first example embodiment. In other words, the vibration transmitting unit 114 can transmit a vibration caused by pulsation to the acceleration sensor 100, the vibration being caused in a region of the vibration transmitting unit 114 where the acceleration sensor 100 is not placed.

In addition, since the vibration transmitting unit 114 in the pulse wave measurement device 14 of the fifth example embodiment has a higher vibration transmissibility than the measured site 140, vibrations caused by pulsation and transmitted by the vibration transmitting unit 114 can be less damped, thereby achieving detection of a true pulse wave.

### Sixth Example Embodiment

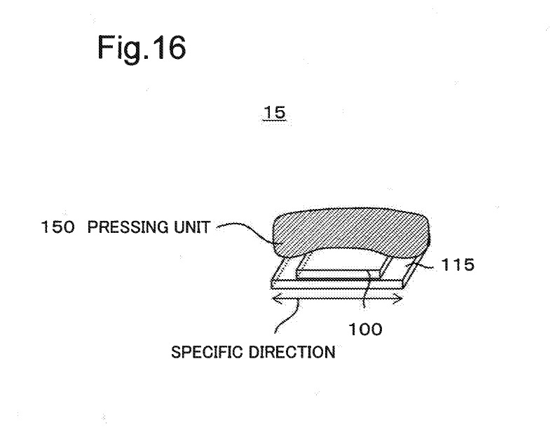

A sixth example embodiment will now be described. FIG. 16 illustrates a configuration of a pulse wave measurement device according to the sixth example embodiment. In the drawing, identical symbols are given to components similar to those in the first example embodiment.

As illustrated in FIG. 16, the pulse wave measurement device 15 according to the sixth example embodiment includes the acceleration sensor 100, the vibration transmitting unit 110, and a pressing unit 150. That is, inclusion of the pressing unit 150 is the only difference from the first example embodiment, and the present example embodiment is otherwise identical to the first example embodiment.

The pressing unit 150 in the pulse wave measurement device 15 of the sixth example embodiment is positioned so that the acceleration sensor 100 and the vibration transmitting unit 115 lie between the pressing unit 150 and a measured site (not illustrated). Changing the amount of a fluid in the pressing unit 150 applies pressure to the pulse wave measurement device 15 to make it more adaptive to the measured site as well as to provide a larger area of contact, thereby achieving detection of a more accurete pulse wave compared with the first example embodiment.

### Seventh Example Embodiment

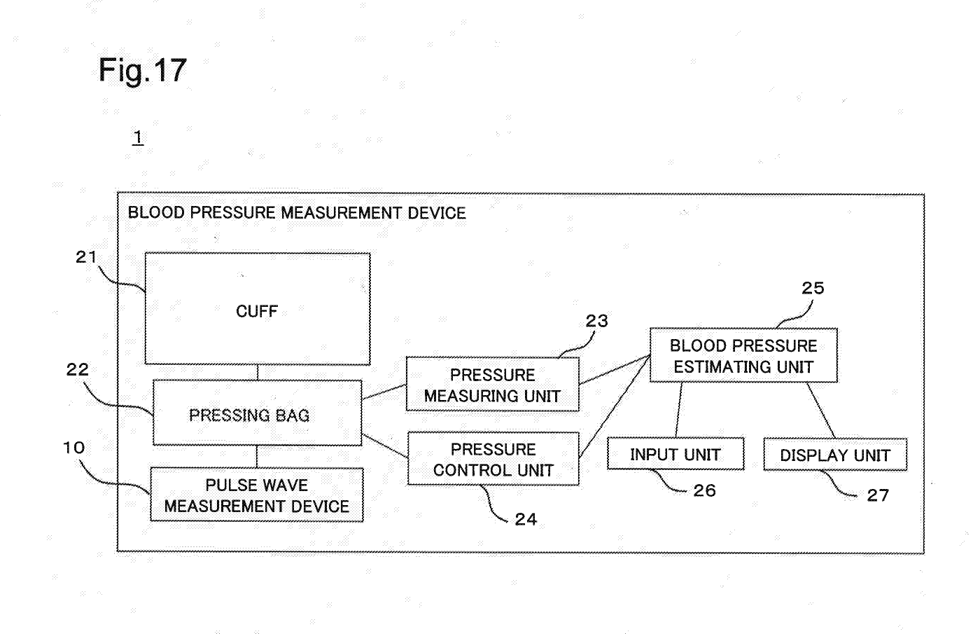

A seventh example embodiment will now be described. FIG. 17 is a block diagram illustrating a configuration of a blood pressure measurement device according to the seventh example embodiment. Specifically, the blood pressure measurement device 1 of the present example embodiment includes a cuff 21, a pressing bag 22 provided along with the cuff 21, at least one pulse wave measurement device 10, a pressure measuring unit 23, a pressure control unit 24, and a blood pressure estimating unit 25. The pressure measuring unit 23 measures internal pressure in the pressing bag 22. The pressure control unit 24 controls internal pressure in the pressing bag 22. The blood pressure estimating unit 25 estimates blood pressure information regarding the subject based on results from the pressure measuring unit 23, the pressure control unit 24, and the pulse wave measurement device 10. The blood pressure measurement device 1 may be configured to further include an input unit 26 that inputs instruction information to the blood pressure estimating unit 25 and a display unit 27 that displays, for example, estimation results provided by the blood pressure estimating unit 25.

The cuff 21 has a strip or ring structure and can be attached on part of a living body, such as an upper arm, a leg, or a wrist.

The pressing bag 22 has a structure capable of containing a fluid (e.g., gas, gel, or liquid) inside. The pressing bag 22 is used to apply pressure to a measured site by containing a fluid inside. The pressing bag 22 may have a single bag or a plurality of bags combining, for example, a gel bag containing a gel with an air bag containing a gas. The pressing bag 22 may optionally have a pump, valve, or the like (not illustrated) for adjusting the amount of a fluid contained in the pressing bag 22.

One or a plurality of pulse wave measurement devices 10 are connected to the pressing bag 22. The pulse wave measurement device 10 measures one or a plurality of pulse waves observed when the amount of a fluid in the pressing bag 22 is changed.

The pressure measuring unit 23 measures internal pressure in the pressing bag 22. In an example, the pressure measuring unit 23 discretizes the measured pressure to transform it into a digital signal (i.e., analog/digital conversion, which is hereinafter referred to as “A/D conversion”). The pressure measuring unit 23 then sends the resulting digital signal as a pressure signal. During the A/D conversion, the pressure measuring unit 23 may extract part of the pressure signal through the use of a filter or the like for extracting a specific frequency. In addition, the pressure measuring unit 23 may amplify the pressure signal so that the signal has a predetermined amplitude, through the use of an amplifier or the like.

The pressure control unit 24 controls internal pressure in the pressing bag 22. Operations of the pressure control unit 24 may include, for example, controlling the amount of a fluid contained in the pressing bag 22 while referring to the pressure signal sent from the pressure measuring unit 23. More specifically, the pressure control unit 24 controls the pump that feeds the fluid to be contained in the pressing bag 22 and valve operations in the pressing bag 22. The pressure control unit 24 controls the pressure applied to the measured site by controlling the internal pressure in the pressing bag 22.

The blood pressure estimating unit 25 estimates blood pressure information based on the pressure signal sent from the pressure measuring unit 23 and at least one pulse wave signal sent from at least one pulse wave measurement device 10. The blood pressure estimating unit 25 may use any known method as the process of estimating blood pressure information. Known methods include, for example, using the oscillometric or Korotkoff method to determine systolic and diastolic blood pressures. Detail descriptions of each of the methods are omitted in the present example embodiment. When estimating the blood pressure information, the blood pressure estimating unit 25 may send a control signal indicating a specific control to the pressure control unit 24.

In the case where the blood pressure measurement device 1 includes the input unit 26, the input unit 26 may include, for example, a measurement start button for starting measurement, a power button, and a measurement cancel button for canceling the ongoing measurement. In the case where the display unit 27 is included, the input unit 26 may further include, for example, a selection button for selecting an item displayed on the display unit 27 (none of the buttons is not illustrated). The blood pressure measurement device 1 starts measurement in response to, for example, an operation on the input unit 26 performed by the subject.

In the case where the blood pressure measurement device 1 includes the display unit 27, the display unit 27 displays, for example, the blood pressure information estimated by the blood pressure estimating unit 25. The display unit 27 may include, for example, a liquid crystal display (LCD), an organic light-emitting diode (OLED), or an electronic paper. In the case where the display unit 27 includes an electronic paper, the electronic paper may be implemented by, for example, a method such as the micro-encapsulation, electronic powder fluid, cholesteric liquid crystal, electrophoresis, or electrowetting method.

The pulse wave measurement device 10 of the present example embodiment is not limited to the pulse wave measurement device 10 of the first example embodiment. As the pulse wave measurement device 10 of the present example embodiment, any pulse wave measurement device described in the individual example embodiments or their variations may be used.

The pressure measuring unit 23, the pressure control unit 24, and the blood pressure estimating unit 25 may also be configured to be connected over a communication network. In this case, control signals, pressure signals, pulse wave signals, and the like are transmitted/received via the communication network. In the case where the blood pressure measurement device 1 of the present example embodiment includes the input unit 26 and the display unit 27, these components may be configured to be connected to other components via any communication network.

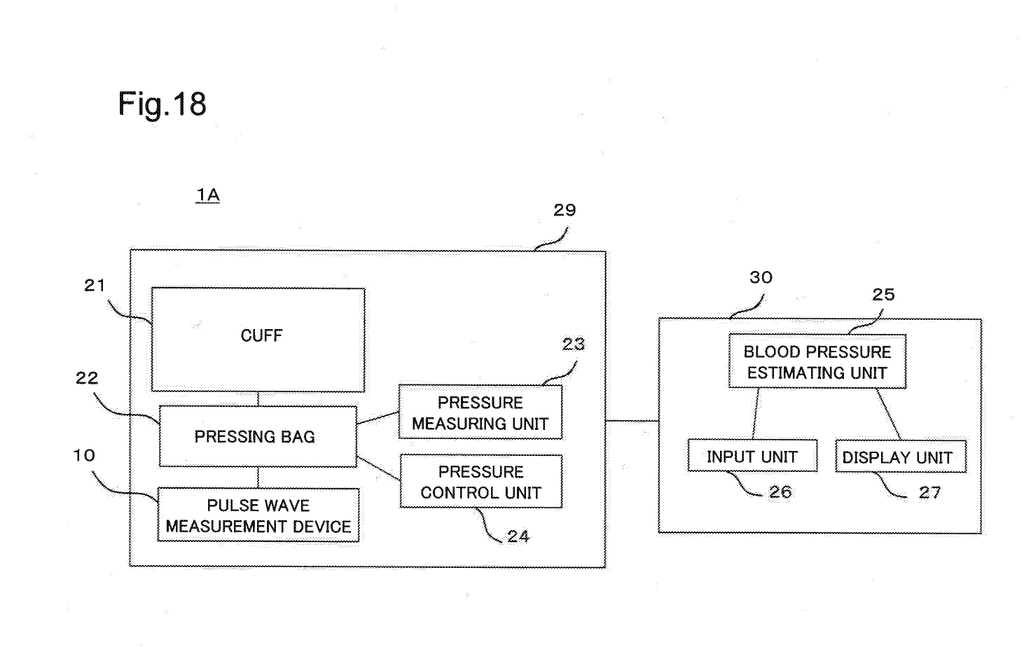

FIG. 18 is a block diagram illustrating a configuration of a blood pressure measurement device according to a variation of the present example embodiment. With reference to FIG. 18, the blood pressure measurement device 1A includes a measurement device 29 and an estimation device 30. The measurement device 29 includes a cuff 21, a pressing bag 22 provided along with the cuff 21, at least one pulse wave measurement device 10, a pressure measuring unit 23, and a pressure control unit 24. The estimation device 30 includes a blood pressure estimating unit 25, an input unit 26, and a display unit 27.

The measurement device 29 and estimation device 30 each uses a wireless communication unit (not illustrated) to be connected to each other via a wireless communication network. Then, a single estimation device 30 may send control signals to a plurality of measurement devices 29 and receive pulse wave signals measured by each of the plurality of measurement devices 29 to estimate blood pressure.

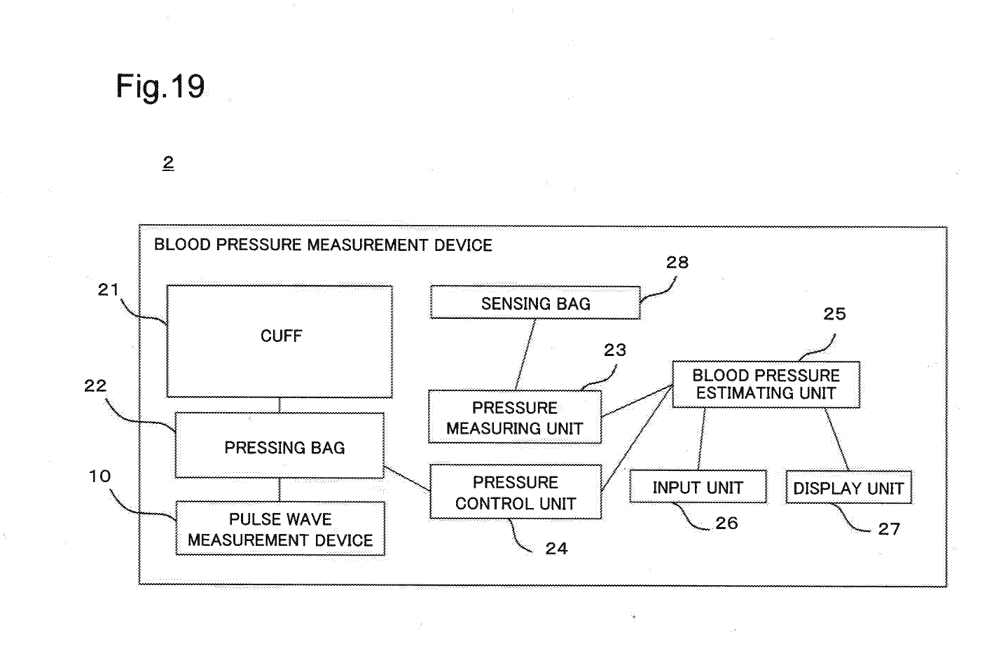

As illustrated in FIG. 19, the blood pressure measurement device 2 of the present example embodiment may include the pressure measuring unit 23 configured to measure pressure other than the internal pressure in the pressing bag 22. For example, the pressure measuring unit 23 may be configured to measure compressing pressure applied to the measured site. In this case, the pressure measuring unit 23 is connected to, for example, a sensing bag 28 to be attached to a surface of the pressing bag 22 facing the living body. The sensing bag 28 is structured to have a shorter length in the specific direction than the pressing bag 22, and thus is capable of obtaining compressing pressure from a limited area in the measured site. Accordingly, blood pressure information can be measured more accurately.

### Eighth Example Embodiment

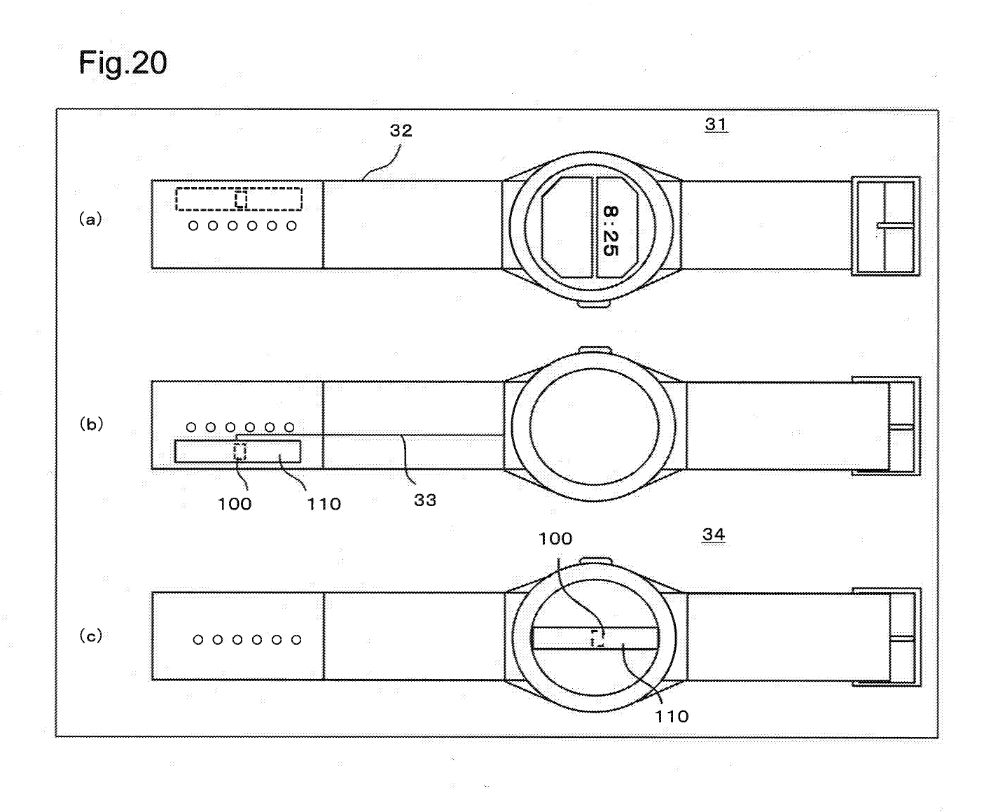

An eighth example embodiment will now be described. FIG. 20 is a schematic diagram illustrating a configuration of a watch according to the eighth example embodiment. In FIG. 20, (a) is a front view of the watch 31, while (b) of FIG. 20 is a back view of the watch 31. In FIG. 20, (c) is a back view of a watch 34.

As illustrated in (a) and (b) of FIG. 20, the watch 31 of the eighth example embodiment includes the pulse wave measurement device 10 (the acceleration sensor 100 and the vibration transmitting unit 110) of the first example embodiment on the back of its band 32.

The pulse wave measurement device 10 is positioned on the band 32 so that, when the watch 31 is worn on a wrist, the vibration transmitting unit 110 in the pulse wave measurement device 10 can catch a vibration caused by pulsation on the inside of a wrist. In other words, the vibration transmitting unit 110 in the pulse wave measurement device 10 is positioned so that the specific direction of the vibration transmitting unit 110 is along the longitudinal direction of the band 32. Relative to the back face of the band 32, the vibration transmitting unit 110 is disposed on the measured site side, and the acceleration sensor 100 is disposed in the thickness direction of the band 32 from the vibration transmitting unit 110.

An electrical signal outputted from the acceleration sensor 100 in the pulse wave measurement device 10 is sent to the main body of the watch 31 through wiring 33 in the band 32. The main body of the watch 31 includes a control unit (not illustrated) and a wireless communication unit (not illustrated). The control unit has a function to transform the electrical signal obtained by the acceleration sensor into pulse wave information and transfer the information to the outside via the wireless communication unit. The band 32 further includes a pressure sensor (not illustrated) located near the pulse wave measurement device 10. An electrical signal outputted from the pressure sensor is sent to the main body of the watch 31 through the wiring 33 in the band 32. The control unit has a function to transform the electrical signal outputted from the pressure sensor into pressure information and give notification of the pressure information.

The following describes measurement of pulse waves performed by the watch 31 according to the eighth example embodiment. First, the user brings the back face of the band 32 equipped with the pulse wave measurement device 10 into contact with the measured site, and applies external pressure to the front face of the band 32 with his/her finger or the like. The external pressure applied to the band 32 is detected by the pressure sensor in the band 32, and then the pressure sensor sends an electrical signal to the main body of the watch 31 through the wiring 33. Depending on the pressure applied to the band 32, the control unit in the watch 31 gives notification by displaying on the watch 31 or producing different sounds so as to obtain a pressure suitable for measuring a pulse wave. Once a pressure suitable for pulse wave measurement is obtained, the control unit in the watch 31 transforms the electrical signal outputted from the pulse wave measurement device 10 into pulse wave information.

In the above-described example, the control unit transforms a signal into pulse wave information, which is then transferred to the outside via the wireless communication unit, but the control unit in the watch 31 may alternatively have a function to estimate blood pressure from pressure information and pulse wave information.

Although the above-described example assumes that a single pulse wave measurement device 10 is provided, the number of pulse wave measurement devices 10 is not limited to one but may be two or more.

In (c) of FIG. 20, a back view of a variation of the watch including the pulse wave measurement device 10 according to the eighth example embodiment is shown. The watch 34 illustrated in (c) of FIG. 20 includes the pulse wave measurement device 10 that is disposed on the back face of the main body of the watch 34. The watch 34 further includes a pressure sensor (not illustrated) located near the pulse wave measurement device 10 on the back face of the main body of the watch 34. The watch 34 according to the variation is different from the watch 31 illustrated in (b) of FIG. 20 in that the pulse wave measurement device 10 and the pressure sensor are located not on the back face of the band 32 of the watch 31 but on the back face of the main body of the watch 34, and other aspects such as configuration and pulse wave measurement are unchanged.

The pulse wave measurement device 10 of the present example embodiment is not limited to the pulse wave measurement device 10 of the first example embodiment. As the pulse wave measurement device 10 of the present example embodiment, any pulse wave measurement device described in the individual example embodiments or their variations may be used.

Although the eighth example embodiment is described, by way of example, with the watches 31 and 34 each including the pulse wave measurement device 10, the eighth example embodiment is not limited to watches, and any portable information processing terminal may be used.

### (Hardware Configuration)

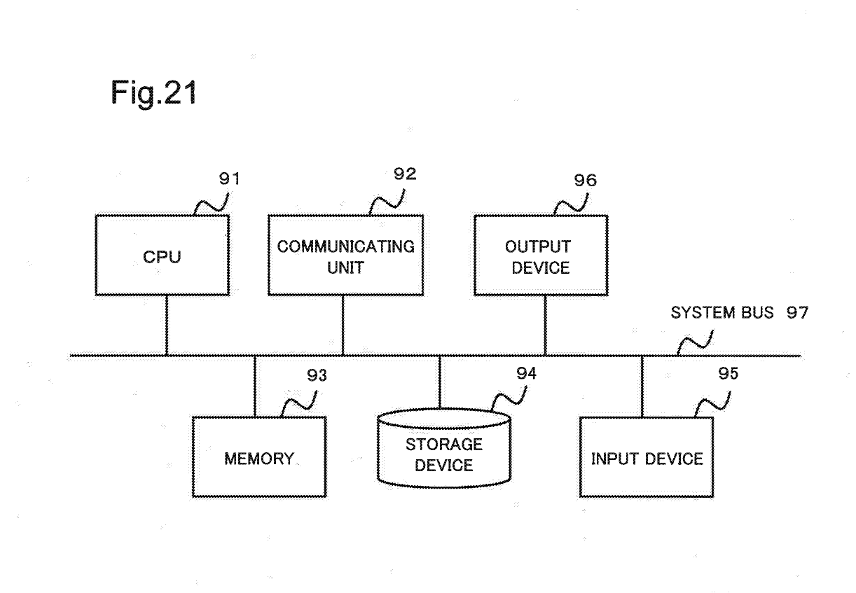

FIG. 21 is a diagram illustrating a hardware configuration for implementing with a computer device the pressure control unit 24 or blood pressure estimating unit 25 in the blood pressure measurement device 1, 1A, or 2 according to the seventh example embodiment, or the control unit in the watch 31 or 34 according to the eighth example embodiment.

As illustrated in FIG. 21, the pressure control unit 24, the blood pressure estimating unit 25, or the control unit includes a central processing unit (CPU) 91, a communication interface (communication I/F) 92 for network connection, a memory 93, and a storage device 94, e.g., a hard disk, for storing a program, and is connected to an input device 95 and to an output device 96 via a system bus 97.

The CPU 91 runs an operating system and controls the blood pressure estimation device of the seventh example embodiment, or controls the control unit in the watch of the eighth example embodiment. In addition, the CPU 91 loads a program and data from, for example, a recording medium mounted on a drive device into the memory 93.

The CPU 91, which corresponds to, for example, controls on the pressure control unit 24 or the blood pressure estimating unit 25 and has a function to process inputted pulse wave vibration signals, executes processing of various functions in accordance with a program.

The storage device 94 may be, for example, an optical disc, a flexible disk, a magneto-optical disk, an external hard disk, or a semiconductor memory. A storage medium being part of the storage device 94 constitutes a non-volatile storage device in which a program is stored. Alternatively, a program may be downloaded from an external computer (not illustrated) connected to a communication network.

The input device 95, which is implemented by, for example, a mouse, a keyboard, a key button, or a touch panel, is used for input operations.

The output device 96, which is implemented by, for example, a display, is used for outputting and confirming information or the like that is produced through processing by the CPU 91.

As seen above, the seventh and eighth example embodiments each are implemented by the hardware configuration illustrated in FIG. 21. However, no specific limitation is imposed on how to implement the individual functional blocks according to the respective example embodiments. That is, each functional block in the blood pressure measurement device or the watch may be implemented by one physically integrated device, or by two or more physically separated devices that are connected to one another with a wired or wireless line.

The present invention has been described with reference to example embodiments (and examples), but the present invention is not limited to these embodiments (and examples). Various modifications of the present invention that could be understood by those skilled in the art may be made to configurations or details of the invention within the scope of the invention.

The whole or part of the above example embodiments can be described as, but is not limited to, the following supplementary notes.

(Supplementary Note 1)

A pulse wave measurement device including: an acceleration sensor that detects a vibration and; a vibration transmitting unit that transmits a vibration caused by pulsation in a measured site, wherein a length of the vibration transmitting unit in a specific direction is longer than a length of the acceleration sensor in a longitudinal direction.

(Supplementary Note 2)

The pulse wave measurement device according to supplementary note 1, wherein the vibration transmitting unit transmits a vibration caused by the pulsation to the acceleration sensor, the vibration being caused in a region of the vibration transmitting unit, and the acceleration sensor being not placed in the region.

(Supplementary Note 3)

The pulse wave measurement device according to supplementary note 1 or 2, wherein a length of the vibration transmitting unit in a direction perpendicular to the specific direction and to a thickness direction of the acceleration sensor is equal to or less than a length of the acceleration sensor in the perpendicular direction.

(Supplementary Note 4)

The pulse wave measurement device according to any one of supplementary notes 1 to 2, wherein the vibration transmitting unit has a curved structure along the specific direction.

(Supplementary Note 5)

The pulse wave measurement device according to any one of supplementary notes 1 to 4, wherein the vibration transmitting unit is deformed along the specific direction under an external pressure of 50 mmHg (6666 Pa) or less applied to the vibration transmitting unit.

(Supplementary Note 6)

The pulse wave measurement device according to any one of supplementary notes 1 to 5, further including a pressurizer that applies pressure to the vibration transmitting unit in a direction toward the measured site.

(Supplementary Note 7)

The pulse wave measurement device according to any one of supplementary notes 1 to 6, wherein a vibration transmissibility of the vibration transmitting unit is higher than a vibration transmissibility of the measured site.

(Supplementary Note 8)

The pulse wave measurement device according to supplementary note 7, further including a band-pass filter that passes a frequency falling within a specific range out of frequencies of an electrical signal or pulse wave information.

(Supplementary Note 9)

The pulse wave measurement device according to supplementary note 1 or 2, wherein the acceleration sensor is placed near a center of the length of the vibration transmitting unit in the specific direction.

(Supplementary Note 10)

The pulse wave measurement device according to supplementary note 1 or 2, wherein the acceleration sensor is placed on an end of the length of the vibration transmitting unit in the specific direction.

(Supplementary Note 11)

The pulse wave measurement device according to supplementary note 1 or 2, wherein the acceleration sensor is placed on a plurality of ends of the vibration transmitting unit.

(Supplementary Note 12)

A blood pressure measurement device including at least one pulse wave measurement device according to any one of supplementary notes 1 to 11.

(Supplementary Note 13)

An information processing terminal including at least one pulse wave measurement device according to any one of supplementary notes 1 to 5, supplementary note 7, and supplementary note 8.

The present application claims priority based on Japanese Patent Application No. 2014-172301 filed on Aug. 27, 2014, the entire disclosure of which is incorporated herein.

### REFERENCE SIGNS LIST

1, 1A Blood pressure measurement device

2 Blood pressure measurement device

10 Pulse wave measurement device

11, 12, 13, 14, 15 Pulse wave measurement device

21 Cuff

22 Pressing bag

23 Pressure measuring unit

24 Pressure control unit

25 Blood pressure estimating unit

26 Input unit

27 display unit

28 Sensing bag

29 Measurement device

30 Estimation device

31 Watch

32 Band

33 Wiring

34 Watch

50 Artery

51 Vibration transmission range

52 Bone

53 Surface layer

91 CPU

92 Communication interface (communication I/F)

93 Memory

94 Storage device

95 Input device

96 Output device

97 System bus

100, 100A, 100B Acceleration sensor

110, 110A, 110B, 110C, 110D, 110E, 110F Vibration transmitting unit

111, 112, 113, 114, 115 Vibration transmitting unit

130 External pressure

140 Measured site

150 Pressing unit

1. A pulse wave measurement device comprising an acceleration sensor that detects a vibration and a vibration transmitter that transmits a vibration caused by pulsation in a measured site,
wherein a length of the vibration transmitter in a specific direction is longer than a length of the acceleration sensor in a longitudinal direction.

2. The pulse wave measurement device according to claim 1, wherein the vibration transmitter transmits a vibration caused by the pulsation to the acceleration sensor, the vibration being caused in a region of the vibration transmitter, and the acceleration sensor being not placed in the region.

3. The pulse wave measurement device according to claim 1, wherein a length of the vibration transmitter in a direction perpendicular to the specific direction and to a thickness direction of the acceleration sensor is equal to or less than a length of the acceleration sensor in the perpendicular direction.

4. The pulse wave measurement device according to claim 1, wherein the vibration transmitter has a curved structure along the specific direction.

5. The pulse wave measurement device according to claim 1, wherein the vibration transmitter is deformed along the specific direction under an external pressure of 50 mmHg (6666 Pa) or less applied to the vibration transmitter.

6. The pulse wave measurement device according to claim 1, further comprising a pressurizer that applies pressure to the vibration transmitter in a direction toward the measured site.

7. The pulse wave measurement device according to claim 1, wherein a vibration transmissibility of the vibration transmitter is higher than a vibration transmissibility of the measured site.

8. The pulse wave measurement device according to claim 7, further comprising a band-pass filter that passes a frequency falling within a specific range out of frequencies of an electrical signal or pulse wave information.

9. The pulse wave measurement device according to claim 1, wherein the acceleration sensor is placed near a center of the length of the vibration transmitter in the specific direction.

10. The pulse wave measurement device according to claim 1, wherein the acceleration sensor is placed on an end of the length of the vibration transmitter in the specific direction.

11. The pulse wave measurement device according to claim 1, wherein the acceleration sensor is placed on a plurality of ends of the vibration transmitter.

12. A blood pressure measurement device comprising at least one pulse wave measurement device according to claim 1.

13. An information processing terminal comprising at least one pulse wave measurement device according to claim 1.
